(19) United States
(12) Patent Application Publication
(10) Pub. No.: US 2024/0327651 A1
(43) Pub. Date: Oct. 3, 2024

(54) COATING AND METHOD FOR COATING A SUBSTRATE

(71) Applicants: ARA-COATINGS GMBH & CO. KG, Nordhorn (DE); BIOGATE AG, Nürnberg (DE)

(72) Inventors: Ralph DOMNICK, Nordhorn (DE); Peter STEINRÜCKE, Nürnberg (DE)

(21) Appl. No.: 18/259,318

(22) PCT Filed: Dec. 23, 2021
(86) PCT No.: PCT/EP2021/087496

§ 371 Date: Feb. 16, 2024

(30) Foreign Application Priority Data
Dec. 29, 2020 (DE) 10 2020 135 064.3

(51) Int. Cl. C09D 5/14; B05D 1/28; C09D 1/00

(52) U.S. Cl. CPC C09D 5/14; B05D 1/28; C09D 1/00

(57) ABSTRACT

A coating in the field of medicine or hygiene includes at least two different portions. At least one of these portions is in the form of a porous layer that can be loaded with liquid and contains silicon as the main component. A further portion is made of multiple layers, specifically a biocide layer and a transport control layer that covers the biocide layer. The porous layer has a silicon proportion (in wt. %) that is at least 1.4 times and at most 5 times the silicon proportion of the transport control layer. At least one porous layer having geometrically defined planar structuring is located on the portion made of the biocide layer and the transport control layer.

The invention relates to a coating suitable for use in the medical technology or hygiene sector. The invention also relates to a method for coating a substrate.

DE 10 2010 054 046 B4 discloses an antibacterial coating which is intended for an implant and contains a layer of copper-titanium nitride. The copper content of the coating is intended to utilise the antibacterial effect of copper without having to accept significant disadvantages with regard to the mechanical properties of the coating, in particular the hardness. Optionally, the coating additionally contains zirconium, which is supposed to achieve a particularly high hardness. In addition to layers containing copper, retardation layers can be provided which do not contain any copper. This should make it possible to control the rate of release of copper ions into the environment. Besides copper, the known coating may contain at least one of the components Ti, Nb, Ta, Cr, Mo, W, Si, Al.

EP 2 281 590 A2 describes a biocorrodible implant with an active coating. This implant has a main body made of a biocorrodible, metal implant material, wherein an active coating and/or cavity filling contains at least one antioxidant substance. The antioxidant substance is, for example, squalene or gallate.

An antimicrobial and non-cytotoxic layered material described in EP 1 790 224 B1 comprises a biocide layer containing silver, copper and/or zinc and a transport control layer covering the biocide layer, the thickness and porosity of which are established such that the biocidal active agent is delivered through the transport control layer in an antimicrobial and non-cytotoxic amount. A base material of the transport control layer may be, for example, a plasma polymer, a sol-gel or a varnish. In either case, the transport control layer has a silicon portion and a carbon portion. For the production of the layered material, vacuum-assisted thin-film processes in particular are proposed in EP 1 790 224 B1.

An antimicrobial and non-cytotoxic layered material which comprises a transport control layer of which the gas permeability for oxygen is 50 to below 100 (cm³ bar)/(day/m²) is known from DE 10 2008 001 014 A1. In addition, an inorganic biocide is also a component of the layered material in this case.

WO 2007/051519 A2 discloses an open-pored biocompatible surface layer for an implant. Here, pores of the surface layer are to be connected to form a coherent pore network. An intermediate layer containing titanium and/or silicon may be provided between the raw surface of the implant and the surface layer.

EP 1 924 300 B1 describes a method for producing a porous coating on a medical implant with structures in the micro- or nanometre range. In this method, temporary particles are deposited on a surface and are removed again in a later method step in order to obtain the desired porosity of the coating. The pores obtained can be at least partially filled with drugs or biologically active ceramic materials.

A mixture described in WO 2007/051806 A1 for a coating process comprises a cross-linkable liquid and a functional component which may contain, inter alia, antimicrobial agents, corrosion inhibitors and colour pigments. The coating produced using the mixture described in WO 2007/051806 A1 is intended to be usable, for example, as a diffusion barrier coating against liquids, gases and/or vapours.

WO 2019/121667 A1 discloses an antimicrobial layered material comprising a layer containing a particulate biocidal active agent and a transport control layer disposed thereon. An after-glow PE CVD process is proposed for depositing the transport control layer. The layered material according to WO 2019/121667 A1 is particularly intended for coating medical devices for human and/or veterinary medical applications. In addition to oxygen and carbon, the transport control layer may contain, for example, silicon or titanium. Silver, copper, zinc or an organic biocide can be considered as a biocidal active gent.

A plastics substrate with a porous layer is known from DE 10 2012 100 288 A1, wherein the porous layer is formed at least partially from the material of the plastics substrate. The volume fraction of the pores is to be greater in a first region of the porous layer than in a second region of the porous layer. The porous layer is to be produced by means of a plasma process.

A method for sintering a porous coating is described, for example, in WO 03/025783 A2. Here, the porous coating is to be built up on a substrate which has openings, in particular in the form of pores. By suitably adjusting the viscosity of a paste from which the coating is obtained, the filling of the pores located in the substrate is to be avoided during the coating process.

WO 2008/040666 A1 discloses a transparent porous SiO₂ coating for a transparent substrate material. Polycarbonate is mentioned as a possible substrate material. A sol-gel process is proposed for the production of the coating.

The object of the invention is to provide silicon-containing coatings which are further developed compared to the aforementioned prior art and which are suitable in particular for the medical technology and hygiene sectors and which are distinguished by properties which can be adapted to specific applications in a variety of ways.

According to the invention, this object is achieved by a coating having the features of claim 1. Likewise, the object is achieved by a method for coating a substrate according to claim 16. The embodiments and advantages of the invention explained below in conjunction with the coating method also apply, mutatis mutandis, to the device, i.e. the coating located on any substrate, and vice versa.

The coating comprises at least two different sub-regions, wherein at least one of these sub-regions is in the form of a porous layer which can be loaded with liquid and contains silicon as the main component, and a further sub-region is constructed in multiple layers, namely from a biocide layer and a transport control layer covering the biocide layer, and wherein the porous layer has a silicon fraction (in % by weight) which is at least 1.4 times and at most 5 times the silicon fraction of the transport control layer.

The different silicon fractions in the various sub-regions of the coating contribute significantly to sub-region-specific different properties. In particular, the transport control layer ensures that material is released from the biocide layer only slowly, while the release of a substance, in particular in liquid form, located in the porous layer, which is defined as the first sub-region, occurs at a comparatively high release rate. The porous layer is in particular a superhydrophilic layer. The superhydrophilic property of the layer means that a drop of water applied to the layer dissipates immediately, i.e. the contact angle is 0°.

Generally, sub-regions of different composition are created on a substrate to be coated. In a first step, a multi-layer sub-region is created, which consists of a biocide layer and a transport control layer covering the biocide layer. In a further step, a laser transfer layer is deposited by placing a carrier at least largely coated with silicon in front of the substrate and then irradiating it by laser in a geometrically defined manner.

The porous, liquid-loadable layer is produced with geometrically defined two-dimensional structuring on the sub-region constructed from the biocide layer and the transport control layer. Optionally, several sub-regions of the coating are formed as porous, liquid-loadable layers containing silicon as the main component. These sub-regions, which are defined as sub-regions of the first type, can be arranged next to each other or at least partially on top of each other.

In the latter case, for example, it is possible to arrange the layers of the first type in an intersecting pattern. Likewise, the porous layers suitable for holding liquid can describe a line pattern. In this case, in one possible embodiment, the total length of the sub-region describing a line pattern is at least eight times the square root of the total area (in cm²) of the coated area, wherein, for example, less than half of the total area of the substrate is coated in the form of the line pattern.

In a preferred embodiment, the porous layer is a laser transfer layer. For the technical background, reference is made by way of example to the documents DE 10 2018 109 337 A1 and WO 2016/055166 A2. A laser transfer layer is produced by placing a transparent film coated with the material to be transferred, in this case silicon, on the substrate and then exposing it to pulsed laser radiation.

The pulsed laser radiation acts in particular in the form of a screened pattern on the transparent film, wherein a solid object, in particular a glass plate, may also be used instead of a film. In any case, the rastered laser radiation can produce a porous superhydrophilic layer, which in a pattern corresponding to the raster of the laser radiation has spaced-apart, approximately point-shaped regions of low roughness and thickness. Between these regions, which are also referred to as laser spots, there is a generally net-shaped intermediate region, which is also attributable to the described porous layer and is also formed mainly by silicon deposited on the substrate and which has a comparatively large roughness and thickness. In particular, the layer thickness of the net-shaped intermediate region is at least three times the layer thickness of the laser spots.

Different sub-regions of the coating may differ from each other with respect to at least one of the parameters constituted by average pore size, porosity, hydrophilicity, pH value, charge, polarity, layer thickness of the porous layer and microbial properties, wherein different properties can be achieved, among other things, by varying laser settings during laser transfer. Here, at least one of the sub-regions may have a gradient of at least one of the aforementioned parameters along its surface. For example, several similar, flat sub-regions, which each have a gradient with respect to a parameter, can be placed directly next to one another, wherein edges of the sub-regions which differ from one another to the greatest extent with respect to the parameter in question adjoin one another.

According to a possible development, within one and the same sub-region, variations are given with regard to several parameters, wherein patterns differing from one another are formed by these variations. In particular, periodic, continuous or discontinuous changes of the relevant parameter along the surface of the sub-region are given by each of the patterns, wherein the period lengths of the different patterns differ from each other.

The coating properties, which change several times on the surface, in particular in a regular pattern or in several superimposed patterns, have the particular purpose of suppressing the spread of germs, which are typically adapted to a specific environment. This plays a role, among other things, in the surface treatment of objects that are used under special hygiene conditions, in extreme cases under clean room conditions.

The coating can be used in a medical implant, for example. The coating is also suitable for covering wounds, wherein in this case the coating can be on a textile material. In general, the coating is suitable for human medical applications as well as for veterinary medical applications.

A particular advantage of the coating is that, provided it is accessible from the outside, it can be recharged with liquid practically as often as desired. In the simplest case, a soaked cloth is merely wiped over the object to be charged with liquid substance, for example a disinfectant liquid, whereby the object absorbs the liquid in its porous structure. On the other hand, the liquid is released over a comparatively long period of time, typically several days, wherein the rate at which the liquid is released into the environment can be controlled over a wide range by adjusting parameters of the coating and is not necessarily uniform over the entire surface of the coated product.

Several exemplary embodiments of the invention are explained in greater detail below with reference to a drawing, in which:

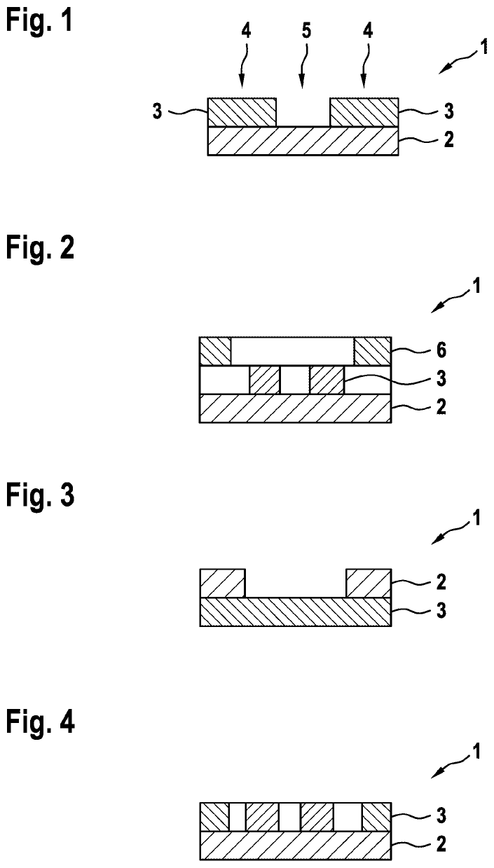

FIG. 1 to 4 show different coating variants on the basis of schematic sectional views,

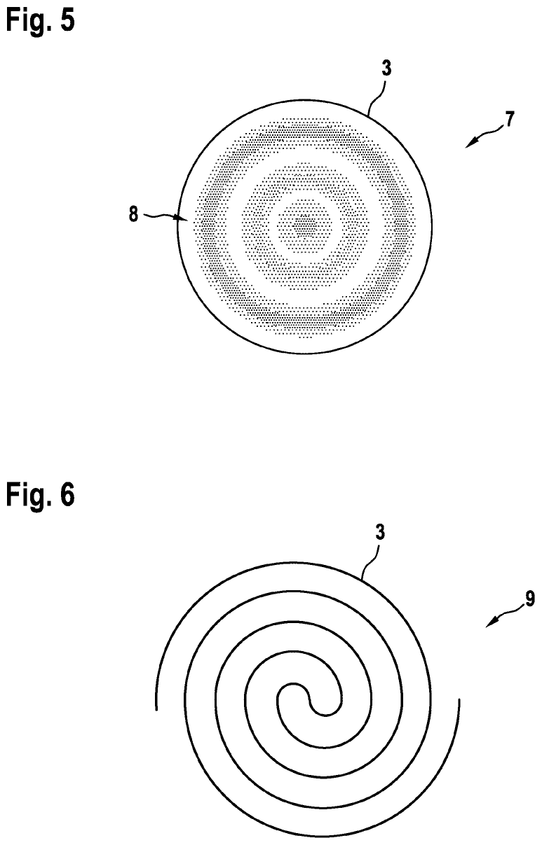

FIG. 5 to 7 show different coating patterns in plan view,

FIG. 8 shows a first example of a coating with sub-regions that are not uniform in design,

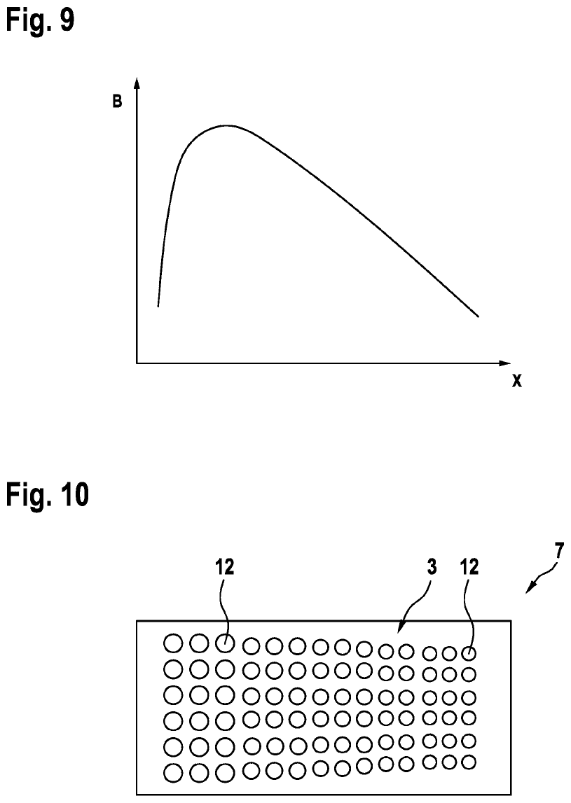

FIGS. 9 and 10 show a porous coating surface with non-uniform pore diameter together with the associated diagram illustrating the loading density on the coating,

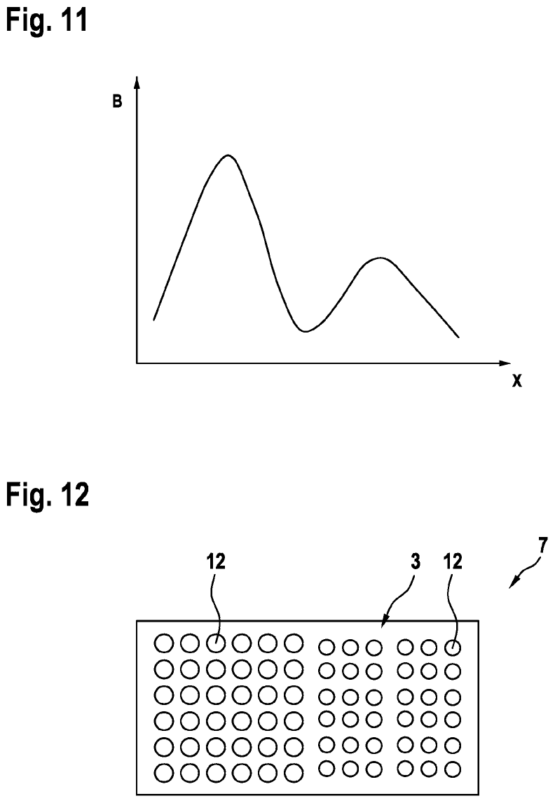

FIGS. 11 and 12 show a further example of an inhomogeneously constructed, loadable coating surface in representations analogous to FIGS. 9 and 10,

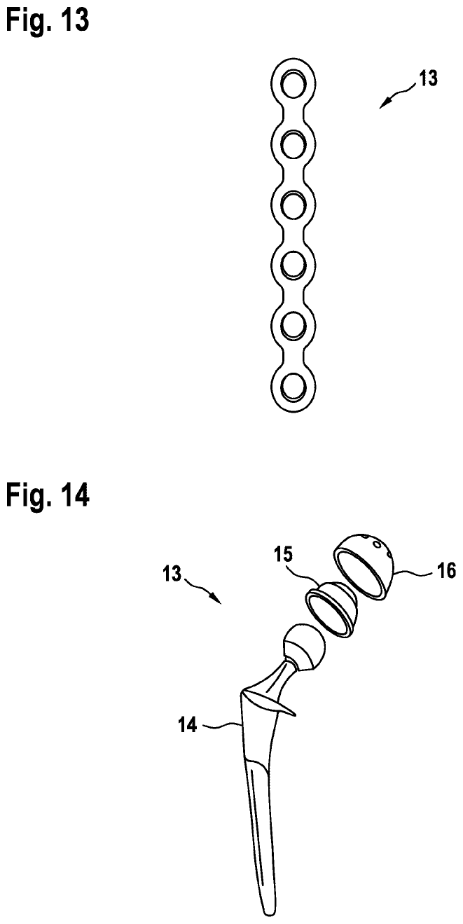

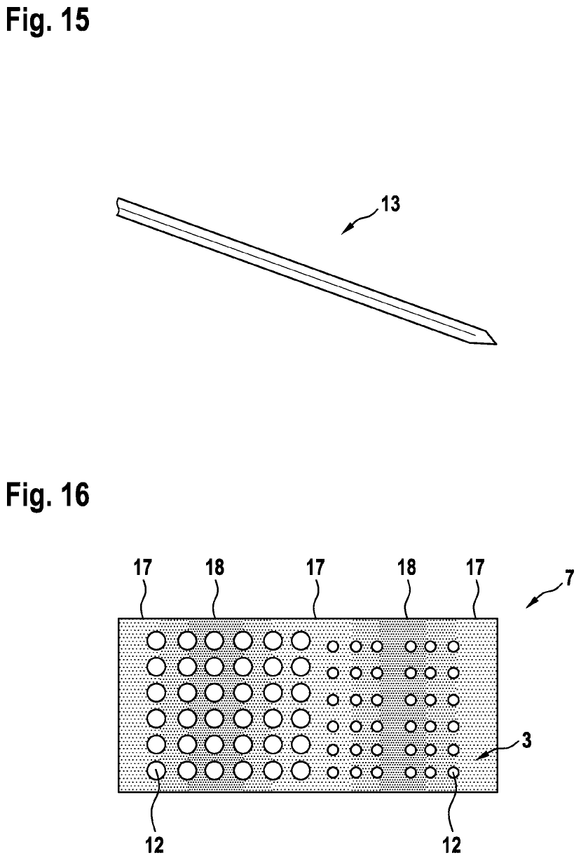

FIG. 13 to 15 show various coated medical products,

FIG. 16 shows a coating area within which different parameters change in different ways in the longitudinal direction of the coating,

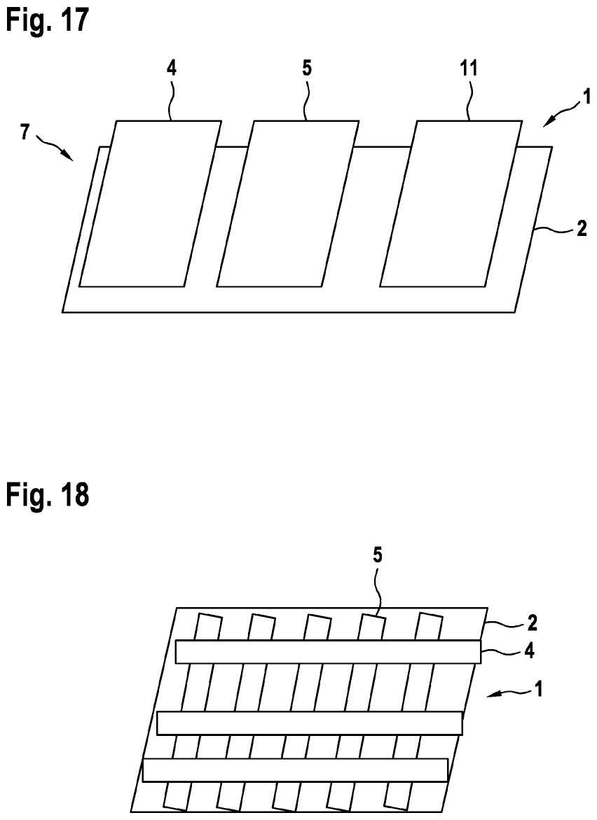

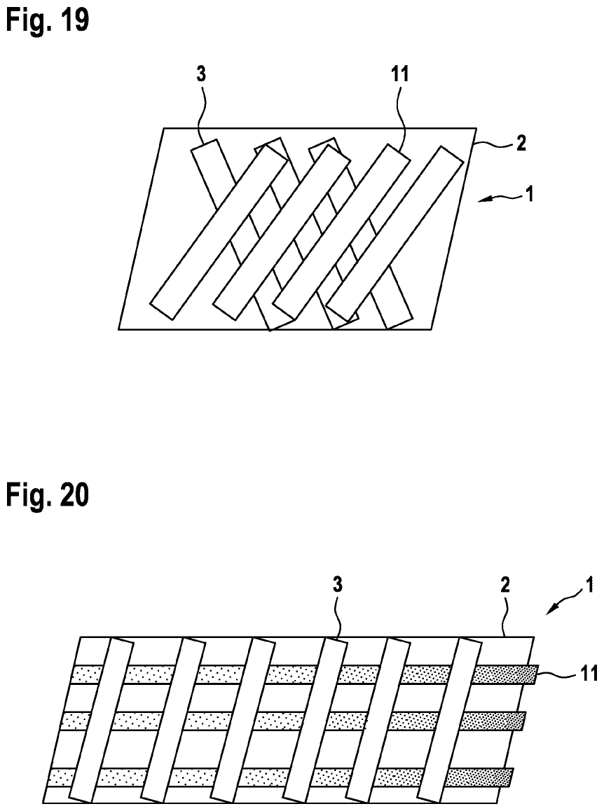

FIG. 17 to 20 show multi-coated surfaces layered in various ways,

FIGS. 21 and 22 show the coating of a workpiece with the aid of a heat shrink tube,

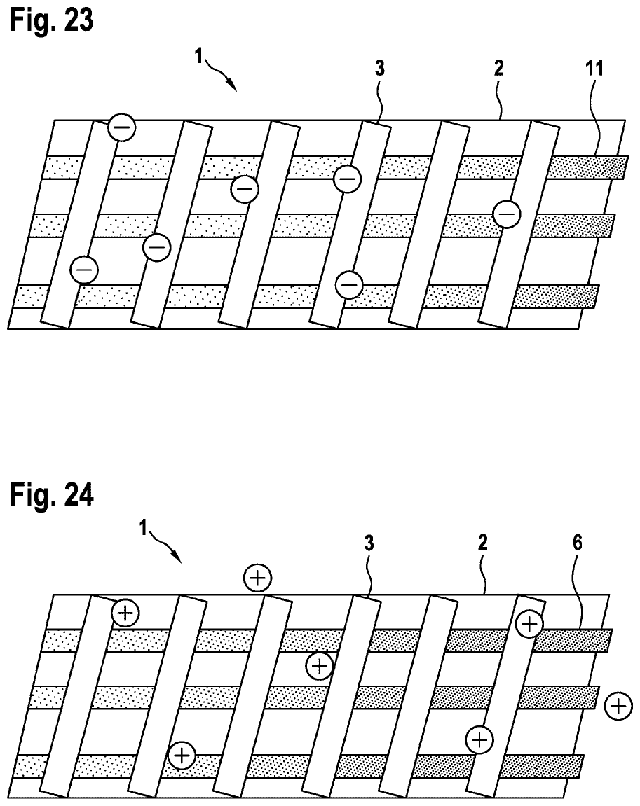

FIGS. 23 and 24 show structured coated surfaces with symbolised electrical charges, and

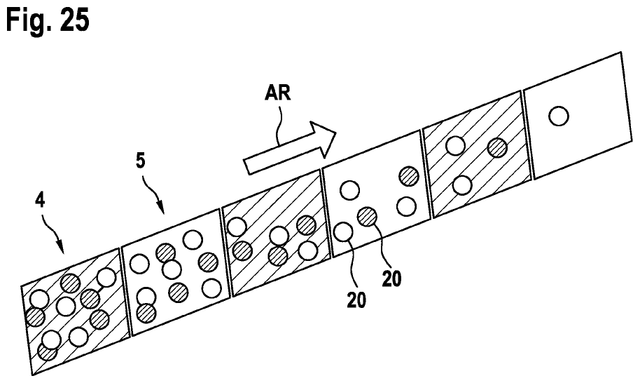

FIG. 25 shows the spreading of fabrics along a surface coated in an alternating pattern.

Unless otherwise stated, the following explanations refer to all exemplary embodiments. Principally comparable components or geometric structures are denoted by the same reference signs in all figures.

A coating generally denoted by the reference sign 1 comprises a layer 2 containing a biocide and at least one porous, superhydrophilic layer 3. The coating 1 is located on a workpiece generally denoted by 7, on the surface of which various partial surfaces 4, 5, 11 can be distinguished from one another.

The at least one porous, superhydrophilic, silicon-based layer 3 covers the biocide-containing layer 2 at least partially in most cases. The reverse layer order is given in the case of FIG. 3. The layer 3 comprises a transport control layer in addition to the biocide in a manner known in principle from EP 1 790 224 B1, but not shown in detail. For example, a PVD process is used to deposit the layer 2. In the exemplary embodiments, the biocide contained in the layer 2 is an inorganic active agent, in particular silver or a substance containing silver. The biocidal active agent is present in the layer 2 in granular form, wherein the average grain size of the primary particles lies preferably in the range from 5 nm to 100 nm.

In contrast to layer 2, layer 3 is applied to the workpiece 7 by laser transfer. In the designs according to FIGS. 1 and 4, the porous superhydrophilic layer 3 is in a single layer; in the design according to FIG. 2, it is in several layers on top of layer 2. In the latter case, the additional porous superhydrophilic layer is denoted 6.

Various ways of distributing at least one porous superhydrophilic layer 3, 6 on the surface of the workpiece 7 are illustrated in FIGS. 5 to 7. According to FIG. 5, a pattern of concentric circles 8 is formed on the substrate surface by the layer 3, wherein coating parameters within the layer 3, as indicated by a raster in FIG. 5, change continuously several times from the centre of the disc-shaped workpiece 7 outwards, so that the image of a sound wave emanating from a point source is obtained. Each maximum of this pattern 8, which is reminiscent of a sound wave, signifies a maximum of a specific product property of the layer 3. The different rasters of the two concentric rings, not sharply delimited, which can be seen in the arrangement according to FIG. 5 signify that a different parameter of the coating 1 has a maximum in each of these two annular disc-shaped regions. The variation of surface properties on the workpiece 7, formed in a geometrically defined manner, plays a role in particular with regard to a spreading of substances taking place on the surface, as will be explained with reference to a further exemplary embodiment.

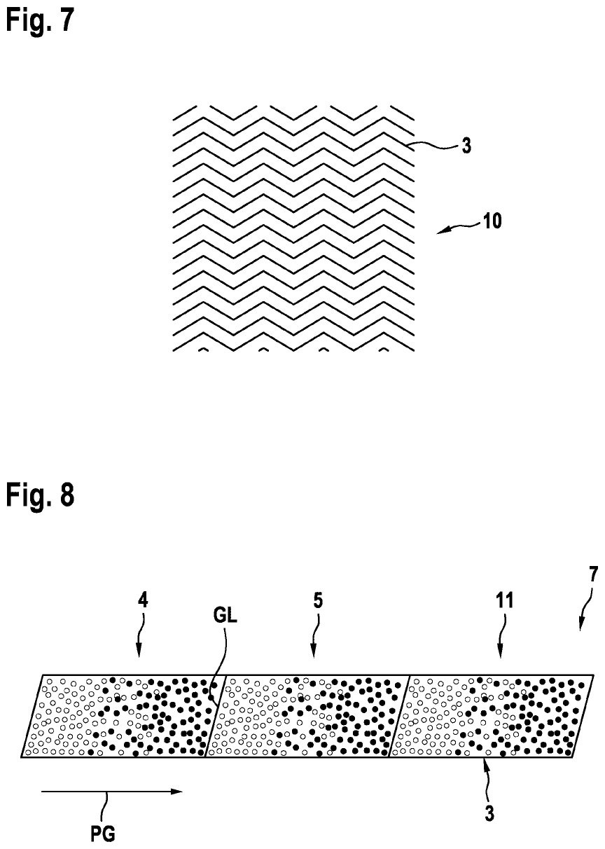

In the exemplary embodiments shown in FIGS. 6 and 7, the porous superhydrophilic layer 3 is applied to the workpiece 7 in the form of a spiral pattern 9 or a zigzag pattern 10. In both cases, intermediate regions are free of the layer 3, so that there are numerous transitions between different coating regions. In the embodiment according to FIG. 8, different partial areas 4, 5, 11 are placed directly next to each other in a row. Within each sub-area 4, 5, 11 a parameter gradient PG is given, which in the present case means a charge gradient, i.e. a continuous transition between “charged” and “uncharged”. While the charge changes only gradually within each sub-area 4, 5, 11, a sudden change occurs at the boundary lines GL between the sub-areas 4, 5, 11. It is precisely these abrupt changes at the boundary lines GL that are particularly effective in suppressing the undesired spread of microorganisms. Instead of a charge gradient, the parameter gradient PG can also be a polarity gradient, i.e. a transition from cationic to anionic.

The porous superhydrophilic layer 3 according to FIG. 10 has multiple individual, idealised pores 12, wherein the pore size decreases from left to right in the arrangement according to FIG. 10. The entire layer 3 can be loaded with a liquid, for example a disinfectant solution. FIG. 9 illustrates the corresponding loading density B, wherein the x-direction corresponds to the longitudinal direction of the workpiece 7 according to FIG. 10. It is clearly visible that more liquid can be absorbed in the region of larger pores 12. This relationship also exists in principle in the exemplary embodiment according to FIGS. 11 and 12, wherein in this case there is a different distribution pattern of the pore size compared to FIG. 10.

FIGS. 13 to 15 show examples of various medical implants 13 as workpieces 7, which are provided with the coating 1 in whole or in part. In the case of FIG. 14, individual parts of the implant 13 are denoted 14, 15, 16. All in all, this is an artificial hip joint. The coating 1, which is at least partially on the individual parts 14, 15, 16, has the property of releasing different substances, which are present on the one hand as a biocide in the layer 2 and on the other hand as a loading in the layer 3, at different release rates.

In the embodiment according to FIG. 16, antimicrobial as well as antibiotic and anti-inflammatory properties of the coating 1 play a role. The antimicrobial properties are mainly due to the composition of the layer 2 containing a biocide. As far as the antibiotic and anti-inflammatory properties are concerned, sub-areas 17 of the first type and sub-areas 18 of the second type, which are distinguishable from each other within the porous layer 3, are of importance. The sub-areas 17, 18 placed directly next to each other form a stripe pattern and signify an alternating polarity sequence. Furthermore, different sizes of the pores 12 are given within the layer 3, as already explained with reference to FIG. 12. However, the regular (in the section according to FIG. 16 only simple) change of the pore size is given in further steps as the change of the polarity. Thus, along the extent of the workpiece 7, i.e. in the x-direction, there are different parameter changes on the product surface, each of which takes place with a specific period length, wherein different period lengths are assigned to the different parameters.

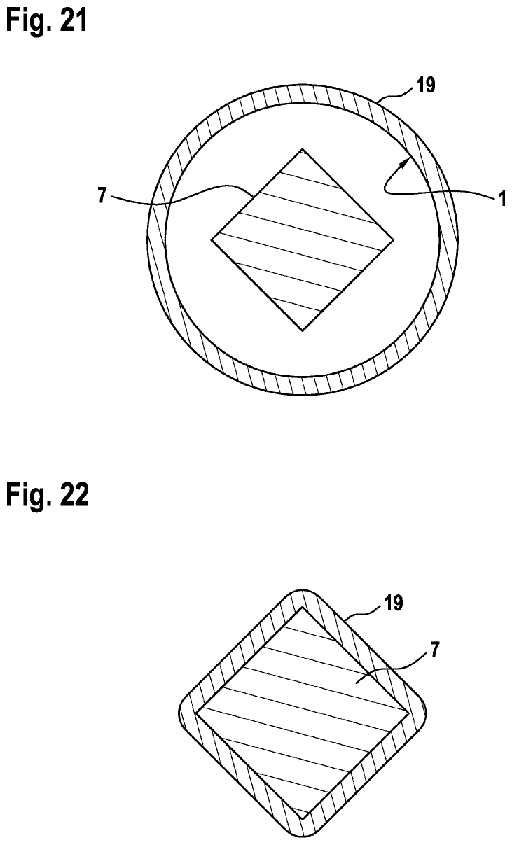

Further possibilities of surface structuring are illustrated in FIGS. 17 to 20. According to FIG. 17, various sub-areas 4, 5, 11, which are provided with the porous layer 3, are loaded with a medicament. In each variant illustrated in FIGS. 18 to 20, several layers of the layer 3 are arranged in an intersecting manner, wherein according to FIG. 20 there is a continuous change of a parameter along three sub-areas 11.

FIGS. 21 and 22 illustrate the application of the coating 1 to the entire surface of a workpiece 7 by means of a shrink sleeve 19, on the inside of which the coating 1 is located. In a first step, the shrink sleeve 19 is pulled over the workpiece 7, which may be irregularly shaped, as is the case, for example, with the individual part 14 of the device according to FIG. 14. The shrink sleeve 19, which must be made of transparent material, is then brought into full-surface contact with the workpiece surface by heating. Subsequently, by irradiating the coating 1 with a laser (not shown), the coating 1 is transferred to the surface of the workpiece 7 at the desired locations, either partially or over the entire surface. Also in this process, as already explained, a variation of the coating properties within the coating surface is achievable in a variety of ways.

The exemplary embodiments according to FIGS. 23 and 24 differ from the exemplary embodiment according to FIG. 20 in that defined polarities are given. Through the different polarities of the layer 3, in the case of FIG. 23 anionic and in the case of FIG. 24 cationic, the release of silver ions, which are located in the layer 2, can be controlled.

In the case of FIG. 25, different sub-areas 4, 5 are placed alternately next to each other, wherein the sub-areas 4 are hydrophilic and the sub-areas 5 are hydrophobic. Symbolically, individual germs 20 are also drawn in FIG. 25, which spread in the spreading direction AR, corresponding to the x-direction. As illustrated in FIG. 25 by a decreasing number of germs 20 per sub-area 4, 5, the constant change between different properties of the sub-areas 4, 5 efficiently counteracts the spreading of the germs 20. In principle, the same effect can be achieved if the sub-areas 4, 5 have a clearly different pH value. A larger area, for example a surface of a medical device or a floor, can, for example, be constructed like a chessboard from different sub-areas 4, 5, which do not differ from each other visibly.

### LIST OF REFERENCE SIGNS

1 coating
2 biocide-containing layer, sub-region
3 porous superhydrophilic layer
4 first sub-area
5 second sub-area
6 further porous superhydrophilic layer
7 workpiece
8 coating pattern: concentric circles
9 spiral pattern
10 zigzag pattern
11 further sub-area
12 pore
13 workpiece
14 individual part
15 individual part
16 individual part
17 sub-area of the first type
18 sub-area of the second type
19 sleeve
20 germ
AR spreading direction
B loading intensity
GL boundary line
PG parameter gradient

1. A coating in the medical technology or hygiene sector comprising at least two different sub-regions (4, 5, 11), wherein at least one of these sub-regions (4, 5, 11) is in the form of a porous layer (3, 6) which can be loaded with liquid and contains silicon as the main component, and a further sub-region (2) is constructed in multiple layers, namely from a biocide layer and a transport control layer covering the biocide layer, characterised in that the porous layer (3, 6) has a silicon fraction (in % by weight) which is at least 1.4 times and at most 5 times the silicon fraction of the transport control layer, and at least one porous layer (3, 6) with geometrically defined two-dimensional structuring is disposed on the sub-region (2) constructed from the biocide layer and the transport control layer.

2. The coating according to claim 1, characterised in that a plurality of the sub-regions (4, 5, 11) are formed as porous, liquid-loadable layers (3, 6) containing silicon as the main component.

3. The coating according to claim 2, characterised in that several of the porous layers (3, 6) are arranged at least partially on top of each other.

4. The coating according to claim 2 or 3, characterised in that the porous layers (3, 6) are arranged in an intersecting pattern.

5. The coating according to any one of claims 1 to 4, characterised in that the sub-region (4, 5, 11) formed as a porous layer (3, 6) describes a line pattern.

6. The coating according to claim 5, characterised in that the total line length of the sub-region (4, 5, 11) describing a line pattern is at least eight times the square root of the total area (in cm²) of the coated area, wherein less than half of the total area is coated in the form of the line pattern.

7. The coating according to any one of claims 1 to 6, characterised in that the porous layer (3, 6) is formed as a laser transfer layer.

8. The coating according to any one of claims 1 to 7, characterised in that the different sub-regions (2, 4, 5, 11) differ from each other with respect to at least one of the parameters constituted by average pore size, porosity, hydrophilicity, pH value, charge, polarity, layer thickness of the porous layer and microbial properties.

9. The coating according to claim 8, characterised in that at least one of the sub-regions (2, 4, 5, 11) has a gradient (PG) of at least one of said parameters along its surface.

10. The coating according to claim 9, characterised in that several similar, flat sub-regions (2, 4, 5, 11), which each have a gradient (PG) with respect to a parameter, are placed directly next to one another, wherein edges of the sub-regions which differ from one another to the greatest extent with respect to the parameter in question adjoin one another.

11. The coating according to any one of claims 8 to 10, characterised in that, within one and the same sub-region (2, 4, 5, 11), variations are given with regard to several parameters, wherein patterns differing from one another are formed by these variations.

12. The coating according to claim 11, characterised in that periodic, continuous or discontinuous changes of the relevant parameter along the surface of the sub-region (2, 4, 5, 11) with different period length are given by each of the patterns.

13. A surface structure in the medical technology or hygiene sector wherein a structuring of the surface counteracting the spread of germs is provided in that numerous geometrically defined sub-regions (2, 4, 5, 11) of the surface placed alternately next to each other represent different ambient conditions for the germs, wherein, due to the multiplicity of borders between the sub-regions (2, 4, 5, 11), a corresponding number of sudden changes in the ambient conditions is provided.

14. Use of a coating according to claim 1 in a medical implant.

15. Use of a coating according to claim 1 for covering wounds, wherein the coating is on a textile material.

16. A method for coating a substrate, wherein sub-regions (2, 4, 5, 11) of different composition are produced on the substrate, specifically, in a first step, a multi-layer sub-region (2), which is constructed of a biocide layer and a transport control layer covering the biocide layer, and, in a further step, a laser transfer layer (3, 6), which is deposited by placing a carrier at least largely coated with silicon in front of the substrate and then irradiating it by laser in a geometrically defined manner.

17. The method according to claim 16, characterised in that a shrink sleeve (19) coated on its inner side is used as carrier and is pulled over the substrate (7) to be coated and, prior to the laser radiation, is brought into full-surface contact with the substrate (7) by heating.

18. The method according to claim 16, characterised in that an expandable sleeve coated on its outer side is used as carrier, wherein this sleeve is introduced into a transparent tube and is inflated, and wherein coating material of the sleeve bearing against the inner wall of the tube is then transferred onto the inner wall of the tube by external laser radiation.
